(19) United States
(12) Patent Application Publication
(10) Pub. No.: US 2014/0127313 A1
(43) Pub. Date: May 8, 2014

(54) PREVENTION AND TREATMENT OF HIV INFECTION

(71) Applicants: Roberto De Jesus Escobar-Gaviria, Medellin (CO); Nicolas Enrique Escobar-Urquijo, Medellin (CO); Luis Felipe Rodriguez-Sanchez, Miami, FL (US)

(72) Inventors: Roberto De Jesus Escobar-Gaviria, Medellin (CO); Nicolas Enrique Escobar-Urquijo, Medellin (CO); Luis Felipe Rodriguez-Sanchez, Miami, FL (US)

(73) Assignee: RONILU DEVELOPMENT CORPORATION, Miami, FL (US)

(21) Appl. No.: 14/129,878

(22) PCT Filed: Jun. 27, 2012
(86) PCT No.: PCT/US12/44315

§ 371 Date: Dec. 27, 2013

Related U.S. Application Data
(60) Provisional application No. 61/502,475, filed on Jun. 29, 2011.

(51) Int. Cl. A61K 35/14

(52) U.S. Cl. CPC A61K 35/14
USPC 424/529

(57) ABSTRACT

A pharmaceutical composition for preventing and treating HIV infection includes an active ingredient made by a process involving a series of intra- and inter-species blood transfers made among male and female animals. The process modifies the components of blood in the animals such that the blood from the last animal in the series of transfers can be used as the active ingredient in a pharmaceutical composition which, when administered to an HIV-infected human subject, can eliminate any detectable HIV in the subject.

### CROSS-REFERENCE

The present application claims priority from U.S. provisional patent application Ser. No. 61/502,475 filed on Jun. 29, 2011.

### FIELD OF THE INVENTION

The invention relates generally to the field of immunology, infectious diseases, virology, and medicine. More specifically, the invention relates to compositions and methods for preventing and treating infections caused by human immunodeficiency virus (HIV).

### BACKGROUND

HIV is a retrovirus that infects cells of the immune system, destroying or impairing their function. As the infection progresses, the immune system becomes weaker, so the person becomes more susceptible to cancer and other infectious agents. The most advanced stage of HIV infection is acquired immunodeficiency syndrome (AIDS). HIV is transmitted through direct contact of a mucous membrane or a bodily fluid containing HIV (e.g., blood, semen, vaginal fluid, or breast milk). The virus is transmitted by unprotected sexual intercourse (anal or vaginal), transfusion of contaminated blood, sharing of contaminated needles, and between a mother and her infant during pregnancy, childbirth and breastfeeding. According to UNAIDS estimates, in 2009, there were about 33.3 million people living with HIV/AIDS, about 2.6 million people were new infected with HIV, and 1.8 million died from AIDS.

Treating HIV infection has proven to be fraught with difficulty. A series of antiretroviral drugs have been developed and used with varying degrees of success. Antiretroviral therapy (ART) can slow the course of the disease, but there is no known cure or vaccine for HIV AIDS. WHO and UNAIDS estimate that at least 14.6 million people were in need of antiretroviral therapy in 2009. As of the end of 2009, more than 30 million infected people were living in low- and middle-income countries, where access to therapy is limited. Alarmingly, only 5.25 million people had access to antiretroviral therapy in these areas.

Highly Active Anti-Retroviral Therapy (HAART) is the current treatment regimen for HIV infection. HAART consists of combinations of nucleoside analogue reverse transcriptase inhibitors plus a non-nucleoside reverse transcriptase inhibitor or a protease inhibitor. HAART has dramatically slowed the progression of the disease, improved the patients' quality of life, and in many cases, significantly lowered their viral load. However, it does not cure the disease, nor does it prevent the recurrence of the disease once the therapy is suspended. Over fifty percent of patients suffer severe side effects such as lipodystrophy, dyslipidemia, insulin resistance, and cardiovascular risks due to HAART. Impediments to the success of this long-term therapy include: non-adherence and non-persistence in the drug regimen, insufficient access to medical care, psychiatric disease, drug abuse, and lack of social support. Even with HAART, HIV patients suffer from cancers, kidney disease, cardiovascular disease, osteoporosis, neural disorders, and it is not clear if these symptoms are due to the disease or to the HAART treatment itself.

Drug resistance to HAART is a new development that hinders treatment success. Surveys conducted in 20 countries showed an overall transmitted drug resistance rate of 3.7%. Drug resistant strains of HIV continue to replicate in the presence of the drug to which it has become resistant. Insufficient knowledge among patients and health workers, suboptimal adherence to treatment regimens, drug stock-outs, and inadequate patient monitoring mechanisms, are among the many factors leading to treatment failure and eventually drug resistance. Patients then will require a second-line regimen which, on average, is at least six times more expensive than the first-line treatment.

Different therapeutic approaches are under development to target the HIV virus. These include a) capture of the virus by chemical or biological methods (such as phagocytosis); b) damaging proteins essential to viral replication such as gp120, gp41, the matrix, the capsid or the reverse transcriptase; c) using chemicals or proteins to bind the docking protein gp120 or the CD4 receptors; d) inhibiting biochemical pathways; and e) targeting the infected cells. Most of these are at the experimental stages and very few have reached clinical trials.

Immunotherapeutic approaches have mostly focused on vaccine development. There are several reasons why developing a vaccine for HIV differs from other vaccines, mainly a) that most vaccines protect against the disease itself, but in this case, the HIV virus will be latent for many years before causing signs or symptoms of disease; b) the killed virus is not immunogenic and using a live attenuated virus poses serious risks; c) most other vaccines are protective against an infrequent infection whereas individuals at high risk for HIV infection, will probably encounter the virus very often; and d) HIV infection itself is immunosuppressive.

There are many technical problems in designing HIV vaccines. The difficulties reside in the high variability of the HIV envelope proteins, in which the dominant epitopes are the most susceptible to mutations and to post-translational modifications. The synthetic peptides that are used for vaccine production can assume conformations that differ from their conformation within the native protein. Also, the HIV isolates used for the development of the vaccine are highly variable and comprise many different clades with a high degree of genetic divergence.

Different platforms for HIV vaccines have been developed based on peptides, proteins, dendritic cell vaccines and plasmid/DNA based vaccines. The protein and peptide based vaccines, such as those using gp120, are safe, but have failed to protect individuals from infection. AIDSVAX did not produce an immune response when tested in the USA. It was then re-tested in Thailand under a new regimen, designed RV14, and it gave a modest 26% protection from infection. Vaccines against the Tat protein were shown to induce functional antibodies; to reduce the marked Th1 polarization of anti-Tat immunity seen in natural infection. A recent phase I trial using a detoxified anthrax-derived polypeptide fused to gag protein p24 elicited a modest response in individuals who have an early infection, with a low viral load. Several autologous dendritic cell vaccines have also been attempted, but they elicited an insufficient cytotoxic T cell response.

Among the vector or plasmid /DNA based vaccines, one of the latest vaccine trials is the STEP vaccine, based on three adenoviral vectors expressing three HIV genes (gag, pol and nef). The STEP vaccine failed to protect Ad5 seronegative individuals against HIV infection, and in the case of Ad5 seropositive subjects, there even was an enhanced susceptibility to HIV infection. In the case of the NYVAC vaccine, there was a robust Th1 response to some HIV antigens, but the vaccine had poor gag-specific responses. Other viral vectors such as canarypox, modified vaccinia, virus Ankara, or pox virus have been tested. These DNA based vaccines, have not been as immunogenic in humans as in the animal trials, and they have not gone past phase I trials.

### SUMMARY

The invention relates to the development of an entirely new therapy for preventing and treating HIV infection. The therapy is based on the surprising discovery that a series of intra- and inter-species blood transfers made among male and female animals modifies the components of blood in the animals such that the blood from the last animal in the series of transfers can be used as the active ingredient in a pharmaceutical composition which, when administered to an HIV-infected human subject, can eliminate any detectable HIV in the subject. Within 40-70 days after a single administration of this therapy to several different HIV+ subjects, the virus was not detectable by ELISA or Western blot in any of the subjects.

Accordingly, in one aspect, the invention features a composition for preventing and treating HIV infection. The composition can be prepared according to a process performed by: administering HIV-infected human blood to a female animal of a first mammalian species that has been made pregnant by a male animal of the first mammalian species; administering blood from the male animal of the first mammalian species to a male animal of a second mammalian species differing from the first mammalian species; administering blood from the male animal of the second mammalian species to a female animal of a second mammalian species; after the female animal of a first mammalian species has given birth to an offspring, administering blood from the female animal of the second mammalian species to the offspring; collecting blood from the offspring after it has been administered blood from the female animal of the second mammalian species; and mixing the collected blood (whole blood or subcomponents thereof such as serum or one or more blood cell fractions) with a pharmaceutically acceptable carrier to form the composition.

The HIV-infected human blood can be administered (e.g., intravenously) to the female animal of the first mammalian species made pregnant by a male animal of the first mammalian species at between 3-14 days of the start of the pregnancy. The blood from the male animal of the first mammalian species can be administered (e.g., intramuscularly) to the male animal of the second mammalian species at between 10-50 days of the start of the pregnancy of the female animal of a first mammalian species. The male animal of the first mammalian species can be the one responsible for making the female animal of the first mammalian species pregnant. The blood from the male animal of the second mammalian species can be administered (e.g., intramuscularly) to the female animal of the second mammalian species at between 3-15 days after the blood from the male animal of the second mammalian species is administered to the female animal of the first mammalian species. The blood from the female animal of the second mammalian species can be administered (e.g., intramuscularly) to the offspring at within 7 days after the female animal of the first mammalian species has given birth to the offspring.

In another aspect, the invention features a method of treating a human subject with an HIV infection. This method includes the step of administering to the subject a composition prepared as described above. The composition can be administered to the subject by intravenous infusion. Performance of the method can result in the treated subject's HIV viral load decreasing, or HIV in the subject becoming undetectable by ELISA or Western blot at between 10-120 days after administration of the composition.

Also within the invention is a method for preventing a human subject from becoming infected with HIV. This method includes the step of administering to the subject a composition prepared as described above. The composition can be administered to the subject by intravenous infusion. Performance of the method can result in the development of immunity against HIV in the subject.

In addition, the invention features a use of the composition described above for treating a human subject with an HIV infection or for preventing a human subject from becoming infected with HIV, or for preparation of a medicament for treating a human subject with an HIV infection or for preventing a human subject from becoming infected with HIV.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions will control. In addition, the materials, methods, and examples are illustrative only and not intended to be limiting.

Other features and advantages of the invention will be apparent from the following detailed description, and from the claims.

### DETAILED DESCRIPTION

The present invention encompasses methods and compositions for preventing and treating HIV infection. The below described preferred embodiments illustrate adaptations of these compositions and methods. Nonetheless, from the description of these embodiments, other aspects of the invention can be made and/or practiced based on the description provided below.

### Biological Methods

Methods involving conventional hematological, immunological, and medical techniques are described herein. Hematological methods are generally known in the art and described in Williams Hematology, 8^{th} Ed., McGraw-Hill Professional, 2010 and Clinical Hematology: Theory and Procedures, M. L. Turgeon, Lippincott Williams & Wilkins, Fourth edition (Dec. 7, 2004) Immunological methods are generally known in the art and described in methodology treatises such as Current Protocols in Immunology, Coligan et al., ed., John Wiley & Sons, New York. General methods of medical treatment are described in McPhee and Papadakis, Current Medical Diagnosis and Treatment 2010, 49^{th} Edition, McGraw-Hill Medical, 2010; and Fauci et al., Harrison's Principles of Internal Medicine, 17^{th} Edition, McGraw-Hill Professional, 2008.

### Compositions for Preventing and Treating HIV Infection

Compositions for preventing and treating HIV infection are prepared by a multi-step process involving a series of blood transfers among different species of mammals. In general, the first step of the process involves administering HIV+ blood to a non-human female mammal, and a short time thereafter removing blood from that female mammal, and administering the removed blood to a neonatal mammal of the same species wherein the neonatal mammal has been administered blood from a mammal of a different species. A few to several days later, blood is removed from the neonatal mammal and used as an active ingredient in a formulation for treating or preventing HIV infection in a human subject.

In preferred embodiments of this process, in a first step, a small volume (e.g., 0.5, 1, 2, 3, 4, 5, 6, 7, 8, 9, 10, 20, or 30 ml) of HIV-infected human blood (preferably whole, fresh, and anti-coagulant treated) taken from a male or female (preferably from subject having a very high viral load, free virus in the blood, and not having been treated with anti-viral medication) is administered (e.g., by intravenous injection) to a female animal of a first mammalian species that has been made pregnant by a male animal of the same species about 3, 4, 5, 6, 7, 8, 9, 10, 11, 12, 13, 14, 15, 16, 17, 18, or 19 days into the pregnancy. When referring to blood removed from an animal, the term “fresh” means within 1, 2, 3, 4, 6, 12, 18, or 24 hours after being removed from the animal. About 10-50 days (e.g., 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51 days) after start of the female animal's pregnancy, blood (preferably whole, fresh, and anti-coagulant treated) from the father is then administered (preferably by intramuscular injection) to a male animal of a second mammalian species differing from the first mammalian species (e.g., dog to cat, horse to cow, etc.). Approximately 3-15 (e.g., 2, 3, 4, 5, 6, 7, 8, 9, 10, 11, 12, 13, 14, 15, or 16) days thereafter, blood (preferably whole, fresh, and anti-coagulant treated) from the male animal of a second mammalian species is administered (preferably by intramuscular injection) into a female animal of the second mammalian species. Immediately after or within 0.5, 1, 2, 3, 4, 5, 6, 7, 8, 9, 10, 11, 12, 13, or 14 days after the female animal of a first mammalian species has given birth to an offspring, blood (preferably whole, fresh, and anti-coagulant treated) from the female animal of the second mammalian species is administered (preferably by intramuscular injection) to the offspring. When the offspring is about 2, 3, 4, 5, 6, 7, 8, 9, or 10 days old, blood (preferably whole, fresh, and anti-coagulant treated) is removed from the offspring and used as the active ingredient in the composition for treating or preventing HIV infection. In some cases, the blood used to treat or prevent HIV infection can be separated into subcomponents which are used as the active ingredient for treating or preventing HIV infection. The subcomponents can be an acellular blood fraction (e.g., plasma, serum, or purified proteins from plasma or serum such as whole protein, a gamma globulin fraction, a purified antibody fraction) or a cellular fraction (e.g., white blood cells, peripheral blood mononuclear cells, T cells, NK cells, or monocytes)

### Mammalian Species

Subjects for use in the above process can be any suitable non-human mammals. Preferred mammals include those having an estrous cycle, e.g., felines, canines, equines, swine, rodents, rats, ovines, and caprines. Examples of felines include domestic house cats and wild cats (e.g., lions, tigers, bobcats, cheetah, jaguar, leopard, lynx, ocelot, and serval). Examples of canines include domesticated dogs and wild dogs (e.g., wolves, foxes, and coyotes). Examples of equines include domesticated horses, ponies, mules, zebras, and mustangs. Examples of swine include domesticated pigs (including miniature pigs) and boars. Examples of rodents include mice, rats, hamsters, gerbils, squirrels, beavers, gophers, porcupines, guinea pigs, chipmunks, prairie dogs, and marmots. Examples of ovines include sheep and lambs. Examples of caprines include domestic and wild goats. Other mammals include rabbits, hares, weasels, bears, seals, lower primates (e.g., lemurs and lorises), and mongooses. Chimeric mammals might also be used. The foregoing mammals can be inbred or outbred, male or female, and of any suitable age (e.g., neonatal, 0-7 days old, 0-1 years old, 1-2 years old, 2-5 years old, 5-10 years old, and adult). Whichever animals are used, to avoid possible transmission of pathogenic agents to a patient, the animals are preferably free of any microbiological (e.g., viruses) agents that can be transmitted to and cause disease in a human patient. The animals can be tested to ensure the absence of such pathogens by known methods, or they may be vaccinated against such pathogens.

### Formulations

The blood or blood components (e.g., plasma, serum, white blood cells, buffy coat cells, PBMCs, NK cells, T lymphocytes, B lymphocytes, macrophages, or monocytes) forming the active ingredient can be diluted (e.g., 1:500, 1:250, 1:100, 1:50, or 1:25; vol:vol) in a pharmaceutically acceptable carrier (e.g., Hartmann's solution) prior to use. A list of pharmaceutically acceptable carriers, as well as pharmaceutical formulations, can be found in Remington's Pharmaceutical Sciences, a standard text in this field, and in USP/NF. Other substances may be added to the compositions and other steps taken to stabilize and/or preserve the compositions, and/or to facilitate their administration to a subject. For example, anti-coagulants can be added to prevent clotting and antibiotics (e.g., a broad spectrum antibiotic such as trimethoprim and sulfamethoxazole) can be included to reduce the chance of a bacterial infection. Other ingredients that might be included are fucus vesiculosus, capsacin, and iodized sulfur.

In one example, 5 ml of the blood that acts as the active ingredient is added to 500 ml of Hartmann's solution just prior to infusion into a patient. A broad-spectrum antibiotic, fucus vesiculosus, capsacin, and iodized sulfur can be added. To prevent adverse reactions, a corticosteroid such as hydrocortisone and one or more antihistamines such as diphenhydramine can be added.

### Methods of Treating an HIV Infection

In another aspect, the invention features a method of treating an HIV infection which involves the step of administering a pharmaceutical composition including the active ingredient blood described above to an HIV-infected human subject. In an exemplary protocol, an HIV-infected human subject to be treated is instructed to fast before administration of the active ingredient. A central line catheter is placed in the patient in case treatment of adverse reactions is required. About twenty minutes before administration of the pharmaceutical composition, the patient is given diphenhydramine and hydrocortisone succinate. Five ml of the active ingredient blood is diluted in 500 ml of Hartmann's solution just before administration. The resulting pharmaceutical formulation is then administered to the patient using a peripheral vein macro drip infusion pump. Any side effects are managed symptomatically.

Any human subject (e.g., males, females, adults, and children) who is infected with HIV might be treated as described above. Preferably, pregnant women are not treated by this method until after having given birth. The anti-viral effects of this treatment may be passed onto children from breastfeeding from a woman who has previously been treated (i.e., factors in the milk can prevent or treat HIV infection).

### Methods of Preventing an HIV Infection

In another aspect, the invention features a method of preventing an HIV infection which involves the step of administering a pharmaceutical composition including the active ingredient blood described above to a human subject not infected with HIV. In an exemplary protocol, the subject is instructed to fast before administration of the active ingredient. A central line catheter is placed in the subject in case treatment of adverse reactions is required. About twenty minutes before administration of the pharmaceutical composition, the subject is given diphenhydramine and hydrocortisone succinate. Five ml of the active ingredient blood is diluted in 500 ml of Hartmann's solution just before administration. The resulting pharmaceutical formulation is then administered to the subject using a peripheral vein macro drip infusion pump. Any side effects are managed symptomatically.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A composition for treating HIV infection, the composition being prepared according to a process comprising:
administering HIV-infected human blood to a female animal of a first mammalian species that has been made pregnant by a male animal of the first mammalian species;
administering blood from the male animal of the first mammalian species to a male animal of a second mammalian species differing from the first mammalian species;
administering blood from the male animal of the second mammalian species to a female animal of a second mammalian species;
after the female animal of a first mammalian species has given birth to an offspring, administering blood from the female animal of the second mammalian species to the offspring;
collecting blood from the offspring after it has been administered blood from the female animal of the second mammalian species; and
mixing the collected blood or a subcomponent thereof with a pharmaceutically acceptable carrier to form the composition.

2. The composition of claim 1, wherein the HIV-infected human blood is administered to the female animal of the first mammalian species made pregnant by a male animal of the first mammalian species at between 3-14 days of the start of the pregnancy.

3. The composition of claim 2, wherein the HIV-infected human blood is administered to the female animal of the first mammalian species intravenously.

4. The composition of claim 1, wherein the blood from the male animal of the first mammalian species is administered to the male animal of the second mammalian species at between 10-50 days of the start of the pregnancy of the female animal of a first mammalian species.

5. The composition of claim 4, wherein the blood from the male animal of the first mammalian species is administered to the male animal of the second mammalian species intramuscularly.

6. The composition of claim 4, wherein the male animal of the first mammalian species was responsible for making the female animal of the first mammalian species pregnant.

7. The composition of claim 1, wherein the blood from the male animal of the second mammalian species is administered to the female animal of the second mammalian species at between 3-15 days after the blood from the male animal of the second mammalian species is administered to the female animal of the first mammalian species.

8. The composition of claim 7, wherein the blood from the male animal of a second mammalian species is administered into the female animal of the second mammalian species intramuscularly.

9. The composition of claim 1, wherein blood from the female animal of the second mammalian species is administered to the offspring at within 7 days after the female animal of the first mammalian species has given birth to the offspring.

10. The composition of claim 9, wherein blood from the female animal of the second mammalian species is administered to the offspring intramuscularly.

11. A method for treating an HIV infection in a human subject, the method comprising the step of administering to the subject a composition prepared according to a process comprising:
administering HIV-infected human blood to a female animal of a first mammalian species that has been made pregnant by a male animal of the first mammalian species;
administering blood from the male animal of the first mammalian species to a male animal of a second mammalian species differing from the first mammalian species;
administering blood from the male animal of the second mammalian species to a female animal of a second mammalian species;
after the female animal of a first mammalian species has given birth to an offspring, administering blood from the female animal of the second mammalian species to the offspring;
collecting blood from the offspring after it has been administered blood from the female animal of the second mammalian species; and
mixing the collected blood or a subcomponent thereof with a pharmaceutically acceptable carrier to form the composition.

12. A method for preventing an HIV infection in a human subject, the method comprising the step of administering to the subject a composition prepared according to a process comprising:
administering HIV-infected human blood to a female animal of a first mammalian species that has been made pregnant by a male animal of the first mammalian species;
administering blood from the male animal of the first mammalian species to a male animal of a second mammalian species differing from the first mammalian species;
administering blood from the male animal of the second mammalian species to a female animal of a second mammalian species;
after the female animal of a first mammalian species has given birth to an offspring, administering blood from the female animal of the second mammalian species to the offspring;
collecting blood from the offspring after it has been administered blood from the female animal of the second mammalian species; and
mixing the collected blood or a subcomponent thereof with a pharmaceutically acceptable carrier to form the composition.
